(19) United States
(12) Patent Application Publication
(10) Pub. No.: US 2012/0046462 A1
(43) Pub. Date: Feb. 23, 2012

(54) PROCESS FOR THE SYNTHESIS OF CHIRAL CYCLIC CARBAMATES

(76) Inventors: Aleksander Warm, Arbaz (CH); Miriam Lorenzi, Naters (CH); Gareth Griffiths, Visp (CH)

(21) Appl. No.: 12/741,073

(22) PCT Filed: Apr. 9, 2010
(86) PCT No.: PCT/EP10/02227

§ 371 Date: Nov. 4, 2011

Related U.S. Application Data
(60) Provisional application No. 61/167,906, filed on Apr. 9, 2009.

(30) Foreign Application Priority Data
Apr. 9, 2009 (EP) 09005218.4

(51) Int. Cl. C07D 265/18

(52) U.S. Cl. 544/92

(57) ABSTRACT

The invention is directed to a process for the preparation of a chiral cyclic carbamate of formula

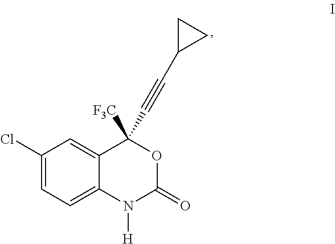

and/or mirror image, and/or a salt thereof,
from the corresponding o-aminobenzyl alcohol and/or salts thereof.

The invention is directed to a process for the preparation of a compound of formula

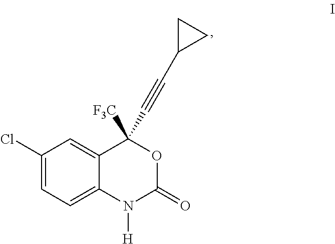

and/or a mirror image and suitable salts thereof.

The chiral cyclic carbamate as depicted in formula I is an important pharmaceutical.

WO-A-98/27073 provides a cyclisation reaction of the corresponding o-aminobenzyl alcohol of the formula

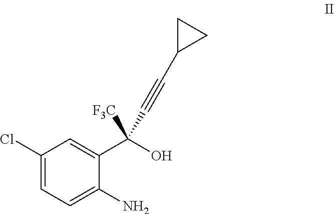

with phosgene in an organic solvent system containing heptanes and tetrahydrofuran. WO-A-98/51676 and WO-A-99/61026 provide a related cyclisation process such an o-aminobenzyl alcohol with phosgene in a biphasic solvent system comprising methyl tert-butyl ether/water or toluene/water in the presence of potassium hydrogen carbonate.

The problem to be solved was to supply an alternative process for the production of the compound of formula I in high yield and quality.

The problem is solved by the process of claim 1.

Provided is a process for the preparation of a compound of formula

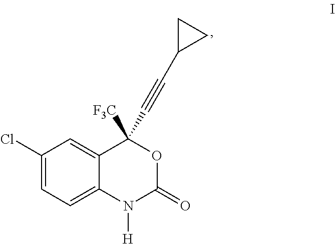

and/or a mirror image and/or a salt thereof,
said process comprising the reaction of
a compound of formula

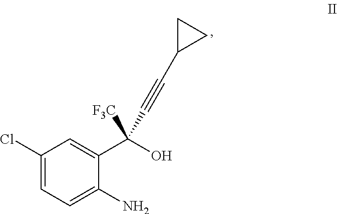

and/or a mirror image and/or a salt thereof, with a phosgene equivalent selected from the group consisting of phosgene, diphosgene or triphosgene, or a mixture thereof; characterized in that the reaction is carried out in the presence of water and at least one water-miscible organic solvent selected from the group consisting of tetrahydrofuran, dioxane, acetonitrile, C₁₋₄-alcohols, dimethoxyethane, diethoxyethane and dimethyl sulfoxide, wherein the pH is in the range of 6 to 11.

Beside phosgene (Carbonyl chloride, COCl₂, CAS No. 75-44-5) there exists two related “dimeric” and “trimeric” compounds, i.e. diphosgene (Trichloromethyl chloroformate, C₂Cl₄O₂, CAS No. 503-38-8) and triphosgene (Bis(trichloromethyl) carbonate, C₃Cl₆O₃, CAS No. 32315-10⁻⁹). It is well known that the latter two, from a chemist's point of view, can be regarded as phosgene equivalents, which are more conveniently to handle but possesses the same reactivity. Phosgene, diphosgene or triphosgene are gaseous, liquid or solid under standard conditions (20° C., 1 bar), respectively. Each compound can be used in chemical reactions neat or dissolved in a suitable solvent. They also can be used as a mixture of two or three. One mol of triphosgene has the same effect then three moles of phosgene, while diphosgene has the same effect then two moles of phosgene. Thus, necessary molar amounts of a mixture can be calculated easily. Diphosgene and triphosgene have the advantage of easier dosing and handling in an undeveloped industrial area. Nevertheless, since diphosgen and triphosgene easily develop phosgene for example even in the presence of humid air, the security measurements have to be on the same high level to protect humans, animals and environment. The workup procedures for removal of excess phosgene (and phosgene equivalents if still present) and organic solvents to facilitate crystallization are preferably carried out as known in the art.

Above pH 11 and below pH 6 increased formation of by-products occurs. Adjustment of the pH can be carried out for example by pre-charging a suitable base in the reaction vessel and/or by controlled addition of a suitable base, preferably by addition of an aqueous sodium and/or a potassium hydroxide solution.

Where more than one organic solvent is present, the at least one water-miscible organic solvent has to act as solubilizer providing control of the pH in the liquid phase. Preferably the mixture is a homogeneous aqueous solution or suspension under standard conditions (20° C., 1 bar). Towards complete conversion of the starting material—i.e. near the end of the reaction—it is possible that the pH may drop below pH 6 due to an excess of phosgene. Therefore, pH control in the range of pH 6 to 11 should be provided until at least 90% conversion. The conversion can be determined quickly by standard methods. Short time excursion of the prescribed pH range during the reaction is possible without being outside the scope of the invention.

The compounds of formula II or mirror images can be obtained for example according to WO-A-98/27073, WO-A-98/51676 or WO-A-99/61026.

In the above described process the configuration on the carbon atom carrying the hydroxy group is maintained.

The reaction can be carried out with the free base of formula II as starting compound or a salt of said base with an inorganic or organic acid. Suitable salts are for example hydro-chlorides, sulfonates, methanesulfonates, oxalates or tartrates. Also useful are non stoichiometric mixtures of the compound of formula II and at least one acid. Usually such mixtures contain excess amounts of acid. A preferred salt is a methanesulfonate, more preferably a mixture containing 1.5 molar equivalents of methanesulfonic acid.

The phosgene equivalents phosgene, diphosgene and triphosgene may be provided in gasous, liquid or solid form or dissolved in an organic solvent. In a preferred embodiment it is provided in gaseous form. In another preferred embodiment it is provided in liquid form. In yet another preferred embodiment it is provided in solid form.

In order to improve workup procedure it may be useful to supply phosgene in slight excess. Preferably the molar ratio of the phosgene equivalent, calculated as monomeric phosgene amount, to the compound of formula II is in a range of 1:1 to 2.5:1, more preferably in the range of 1.1:1 to 1.5:1. Generally, the most preferred molar ratio is about 1.2:1 calculated as phosgene.

The base used in the reaction can be an inorganic or organic base. Examples for inorganic bases are alkali or earth alkali metal carbonates, hydrogen carbonates and hydroxides.

Examples of suitable organic bases are piperidine, C₁₋₄-alkylpiperidines, pyridine, C₁₋₄-alkylpyridines, morpholine or tri-C₁₋₄-alkylamines, wherein any of the alkyl moieties are independently selected from the group consisting of methyl, ethyl, propyl, isopropyl, butyl, isobutyl, sec-butyl and tert-butyl. Using weak bases like alkali or earth alkali metal carbonates, hydrogen carbonates or a combination of different bases with different pK_{b} establishes a buffered system wherein the pH can be easily controlled. Using strong bases like alkali or earth alkali metal hydroxides may require parallel dosage of the phosgene equivalents and the base to maintain the pH in the prescribed range.

Herein the term “C₁₋₄-alkyl” represents methyl, ethyl, propyl, isopropyl, butyl, isobutyl, sec-butyl and tert-butyl.

In a preferred embodiment the weight ratio of water to the organic solvent(s) is in the range from 1.5:1 to 5:1, preferably in the range from 2:1 to 3.5:1.

The most suitable organic solvent comprises tetrahydrofuran and mixtures thereof.

Herein the term “C₁₋₄-alcohol” represents an alcohol selected from the group consisting of methanol, ethanol, propanol, isopropyl alcohol, butanol, isobutanol, sec-butanol, tert-butanol, Cl₃CCH₂OH, CF₃CH₂OH, CH₂═CHCH₂OH or fully alkylated amino C₁₋₄-alcohols such as (CH₃)₂NCH₂CH₂OH.

In a preferred embodiment the reaction is carried out at a temperature from −30 to +40° C. until completion of the reaction, preferably in the range from −30 to +30° C.

### EXAMPLES

In all examples the chirality of the compounds is as directly depicted in formulae I and II. If not otherwise mentioned in examples 1 to 11 the pH is adjusted between 6 and 11 during the addition of phosgene stoichiometrically needed for complete conversion. Because phosgene is readily available for the applicant experiments with other phosgene equivalents then phosgene itself have not been carried out because of the well known equivalence of all three available forms. Adjustment of the pH is carried out by addition of the base pre-charged in the reaction. Heptanes in the meaning of the present invention and the experiments means any mixture of linear and branched heptanes, comprising n-heptane as the major component of at least 50%, preferably of at least 70%, more preferably of at least 90% and even more preferably of at least 95%.

### Example 1

The compound of formula II•1.5 CH₃SO₃H (total weight 85.7 g), the methanesulfonic acid salt comprised 56.82 g (196 mmol) of the compound of formula II and 1.5 molar equivalents methanesulfonic acid (28.28 g, 294 mmol), was suspended in THF (90.5 g) and slowly charged with a solution of sodium carbonate (47.7 g, 450 mmol) in water (293 g). The agitated yellow-orange mixture was cooled to about 12° C. and gaseous phosgene (23.3 g, 236 mmol) was added within 1 h wherein the temperature in the reaction vessel was kept at 7 to 17° C. At the end of the phosgene addition the reaction mixture was additionally agitated for 1 h at 12° C. After the agitation time the conversion of the starting compound was tested (conversion >90%) and a further amount of phosgene (about 100 mmol) was added with subsequent agitation time as described above. After complete conversion (>99.7%) heptanes (375 g) and water (60 g) was added for workup. The mixture was heated to about 30° C. and agitated for 30 min at this temperature. The mixture was allowed to stand for about 30 min and a phase separation was performed. Water (130 g) was added to the organic phase, the mixture was agitated for a short time and again allowed to settle for about 30 min. A second phase separation was performed and heptanes (136.6 g) were added to the organic phase. The organic phase was concentrated by distilling off THF/heptanes (about 205 g) at 150 to 250 mbar and about 45° C. Heptanes (171 g) were added and the distillation was continued. The product crystallization started during the distillation. After obtaining a white suspension heptanes (95 g) was added and distilled off again until complete removal of THF. Finally heptanes (13.7 g) were added and the suspension cooled to −10° C. or lower. The slurry was filtered and the filter cake washed with cold heptanes. After drying 96.8% of compound of formula I (60.0 g, 190 mmol) was obtained.

### Example 2

Example 1 was repeated except using potassium hydrogen carbonate as base (90.1 g, 0.90 mol). Yield: 96.9% of white product was obtained.

### Example 3

Example 1 was repeated except using dioxane as organic solvent (178 g). Furthermore the workup procedure after the second phase separation was modified as follows. The organic phase was concentrated to dryness (20-60° C., <20 mbar). Then heptanes (512 g) were added to the solid residue. The slurry was heated to 45° C. and 205 g of heptanes were distilled off at 160 to 180 mbar and 40 to 50° C. Heptanes (170 g) were added to the slurry and the temperature was increased to 70° C. to dissolve the suspension. Then the solution was cooled to 45° C. with a rate of 1° C./min and then further cooled to −10° C. over a period of 1.5 hours. The slurry was stirred at this temperature for 30 min, filtered and washed with cold heptanes. Yield: 93.4% of off-white to beige product was obtained.

### Example 4

Example 3 was repeated except using dimethoxyethane as organic solvent (149 g). Yield 96.9% of white product was obtained.

### Example 5

The compound of formula II•1.5 CH₃SO₃H (total weight 85.7 g, the methanesulfonic acid salt comprised 56.82 g (196 mmol) of the compound of formula II and 1.5 molar equivalents methanesulfonic acid (28.28 g, 294 mmol), was suspended in THF (90.5 g) and water (185 g). A first portion of 25% aqueous NaOH (51.3 g, 0.321 mol) was added at about 10° C. to adjust the pH between 8 and 9 before the addition of phosgene. Then a parallel dosage of phosgene (23.3 g, 236 mmol) and 25% aqueous NaOH (76.7 g, 0.479 mol) was performed at about 12° C. over 1 hour maintaining the pH between 8 and 9. After completion of the reaction, the workup procedure for removal of organic solvent other than heptanes was carried out as described in example 1. Yield: 96.1% of white product was obtained.

### Example 6

Example 1 was repeated except using sodium hydrogen carbonate as base (75.6 g, 0.90 mol). Yield: 97.2% of white product was obtained.

### Example 7

Example 1 was repeated except using potassium carbonate as base (62.2 g, 0.45 mol). Yield: 97.1% of white product was obtained.

### Example 8

Example 5 was repeated except using triethylamine as base (128.3 g, 1.268 mol), the amount of water (293 g) pre-charged to the reaction and the amount of phosgene (38.9 g, 0.39 mol). Yield: 95.5% of off-white product was obtained.

### Example 9

Example 1 was repeated except using acetonitrile as solvent (133 g) and the amount of phosgene (27.3 g, 0.276 mol). During work up, a total of 360 g of water and 20 g of NaCl were added to perform the second phase separation. Yield: 94.3% of white product was obtained.

### Example 10

Example 1 was repeated except using a THF/diethoxymethane (1:1, v:v) mixture as solvent (146 g). The work up procedure was performed like in example 3. Yield: 95.2% of off-white product was obtained.

### Example 11

Example 1 was repeated except a modified work up procedure as follows. After testing the conversion of the aromatic alcohol, the reaction mixture was heated to about 45° C. The mixture was allowed to stand for about 30 min at this temperature and the aqueous phase was removed. Charcoal (1.2 g) was added to the organic phase and the mixture was agitated for additional 30 min at 50° C. The charcoal was filtered off. The filter cake was washed with THF (13.3 g) and the filtrate was concentrated by about half under vacuum. Then heptanes (512 g) was added maintaining the temperature above 40° C. The organic phase was concentrated by distilling off THF/heptanes at 150 to 250 mbar and about 45° C. The product crystallization occurred during the distillation. After complete removal of THF (about 200 mL of distillate), heptanes (200 mL) was added and the suspension was cooled to −10° C. or lower. The slurry was filtered and the filter cake washed with cold heptanes. After drying 91.2% of compound of formula I (56.5 g, 179 mmol) is obtained.

### Comparison Example 1

The compound of formula II•1.5 CH₃SO₃H (total weight 85.7 g) [The methanesulfonic acid salt comprising 56.82 g (196 mmol) of the propargylic alcohol and 1.5 molar equivalents methanesulfonic acid (28.28 g, 294 mmol)] was suspended in MTBE (217 g) and slowly charged with a solution of potassium hydrogen carbonate (90.1 g, 900 mmol) in water (293 g). The agitated yellow mixture was cooled to about 12° C. and gaseous phosgene (23.3 g, 236 mmol) was added within 1 h wherein the temperature in the reaction vessel was kept at 7 to 17° C. At the end of the phosgene addition the reaction mixture was additionally agitated for 1 h at 12° C. After the agitation time the conversion of the aromatic amino alcohol was tested (conversion >90%) and a further amount of phosgene was added with subsequent agitation time as described above. After complete conversion (min. 99.7%), the mixture was heated to about 30° C. and agitated for 30 min at this temperature. The mixture was allowed to stand for about 15 min and a phase separation was performed. Water (130 g) was added to the organic phase; the mixture was agitated for a short time and again allowed to settle for about 30 min before performing a second phase separation. The organic phase was concentrated to dryness (20 to 50° C., <20 mbar). Then heptanes (512 g) was added to the solid residue. The slurry was heated to 45° C. and 205 g of heptanes are distilled at 160 to 200 mbar and 40 to 50° C. Heptanes (170 g) was added to the slurry and the temperature was increased to 76° C. to dissolve the suspension. Then the solution iwas cooled to 45° C. with a rate of 1° C./min and then further cooled to −10° C. over a period of 1.5 hours. The slurry was stirred at this temperature for 30 min, filtered and washed with cold heptanes. Yield: 98.4% of beige product was obtained. Beside the colour the products doesn't met the specs.

### Comparison Example 2

Comparison Example 1 was repeated except using sodium carbonate as base (47.7 g, 0.45 mol) and the amount of MTBE (126 g). During work up, phase separations were performed at 40° C. The suspension was not dissolved before starting to cool down. Yield: 93.2% of beige product was obtained. Beside the colour the products doesn't met the specs.

1. A process for the preparation of a compound of formula 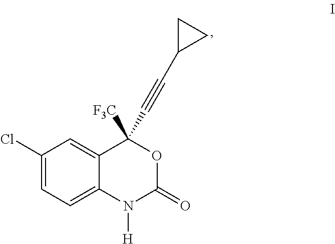
and/or a mirror image and/or a salt thereof,
said process comprising the reaction of
a compound of formula 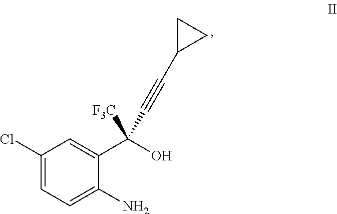
and/or a mirror image and/or a salt thereof,
is reacted with a phosgene equivalent selected from the group consisting of phosgene, diphosgene or triphosgene, or a mixture thereof;
characterized in that the reaction is carried out in the presence of water and at least one water-miscible organic solvent selected from the group consisting of tetrahydrofuran, dioxane, acetonitrile, C₁₋₄-alcohols, dimethoxyethane, diethoxyethane and dimethyl sulfoxide, wherein the pH is in the range of 6 to 11.

2. The process of claim 1, wherein the phosgene equivalent is provided in gaseous form.

3. The process of claim 1, wherein the phosgene equivalent is provided in liquid form.

4. The process of claim 1, wherein the phosgene equivalent is provided in solid form.

5. The process of claim 1, wherein the molar ratio of the phosgene equivalent, calculated as monomeric phosgene amount, to the compound of formula II is in a range of 1:1 to 2.5:1.

6. The process of claim 1, wherein the base is an inorganic or organic base selected from the group consisting of alkali or earth alkali metal carbonates, hydrogen carbonates and hydroxides, piperidine, C₁₋₄-alkylpiperidines, pyridine, C₁₋₄-alkylpyridines, morpholine and tri-C₁₋₄-alkylamines.

7. The process of any of claim 1, wherein the weight ratio of water to the organic solvent(s) is in the range from 1.5:1 to 5:1.

8. The process of claim 1, wherein the organic solvent is tetrahydrofuran.

9. The process of claim 1, wherein the reaction is carried out at a temperature from −30 to +40° C.
